(19) United States
(12) Patent Application Publication
(10) Pub. No.: US 2026/0240637 A1
(43) Pub. Date: Aug. 20, 2026

(54) APPARATUS AND METHODS FOR PROVIDING EMBOLIC PROTECTION DURING A TRANSCATHETER OPERATION

(71) Applicant: Oxford University Innovation Limited, Oxford (GB)

(72) Inventors: Giovanni Luigi DE MARIA, Bedhampton (Hampshire) (GB); Yunlan ZHANG, Oxford (Oxfordshire) (GB); Mohammad ALKHALIL, Killingworth Tyne and Wear (GB); Zhong YOU, Oxford (Oxfordshire) (GB); Yunfang YANG, Oxford (Oxfordshire) (GB)

(21) Appl. No.: 19/164,265

(22) PCT Filed: Mar. 19, 2024
(86) PCT No.: PCT/GB2024/050740

§ 371 Date: Sep. 11, 2025

(30) Foreign Application Priority Data
Mar. 21, 2023 (GB) 2304119.7

(51) Int. Cl. A61F 2/01; A61F 2/24

(52) U.S. Cl. CPC A61F 2/013; A61F 2/2427; A61F 2002/018; A61F 2210/0061

(57) ABSTRACT

In one arrangement, an apparatus comprises an elongate sheath insertable through the vascular system to a target site. A filter arrangement extends from a distal end of the sheath and comprises a porous membrane switchable between a radially contracted state and a radially expanded state. The radially expanded state is such as to span a cross-section of a blood vessel to block debris from travelling past the porous membrane. A filter actuation system hydraulically switches the porous membrane between the radially contracted state and the radially expanded state.

The present disclosure relates to apparatus and methods for providing embolic protection during a transcatheter operation. Disclosed arrangements are particularly suited for use in the aorta during interventional cardiac surgery, such as during Transcatheter Aortic Valve Implantation (TAVI).

TAVI, which is sometimes referred to as TAVR (Transcatheter Aortic Valve Replacement), is a minimally invasive procedure to replace a narrowed aortic valve that fails to open properly, a condition known as aortic valve stenosis. The procedure is being used increasingly due to safety advantages relative to open heart surgery alternatives.

TAVI procedures have been associated with silent ischaemic cerebral embolism and even clinical stroke caused by procedural debris reaching the cerebral vasculature during TAVI prosthesis deployment. In recent large-scale trials, around 5-6% of cases ended up with the occurrence of stroke or a transient ischemic attack, and stroke is associated with a 3.5-fold increase of mortality rates during the first month after TAVI. Aortic debris may be generated, for example, due to the use of large-sized catheters and rigid delivery systems in the calcified native valve and aortic wall. Additional balloon aortic valvuloplasty can be required in TAVI prosthesis deployment, with further risk of dislodgement and embolization of aortic debris and crushed calcified native valves. These embolic particles may enter the bloodstream and embolize to the brain or other organs downstream. Cerebral embolism can lead to occlusion of blood vessels, resulting in neuropsychological deficits, stroke and even death.

The risk of embolism has led to the development of Cerebral Embolic Protection Devices (CEPDs) to protect patients from these risks and thereby improve the outcome of relevant surgical procedures. CEPDs usually comprise a filter or protective sheath designed to capture or deflect emboli traveling to the brain during TAVI procedures in order to prevent embolic debris from reaching the supra-aortic vessels. In a typical TAVI procedure, a CEPD may be inserted percutaneously through the radial or femoral artery before deployment of the TAVI prosthesis. The CEPD may, for example, be placed across the origin of supra-aortic vessels. In some known CEPD devices, filters are deployed that cover some or all the branches of the aortic arch (the brachiocephalic artery, left common carotid artery and left subclavian artery) to prevent debris from entering cerebral circulation. Such devices may protect the brain region vessels, but they cannot prevent debris going to non-cerebral regions. Another type of device aims to provide full circumferential coverage of the aortic arch, protecting all supra-aortic vessels and preventing migration of debris to both cerebral and non-cerebral regions. However, such devices use a free-standing woven mesh filter, which is relatively costly and considerable skills are needed to master deployment of the device.

CEPD devices such as those discussed above provide varying levels of embolic protection and/or can be costly and/or complex to manufacture. They furthermore require a dedicated vascular access to be inserted in addition to the vascular access required to deliver the TAVI prosthesis, which places stress on the patient and complicates the surgical procedure.

It is an object of the present disclosure to at least partly address one or more of the challenges discussed above.

According to an aspect of the invention, there is provided an apparatus for providing embolic protection during a transcatheter operation, the apparatus comprising: an elongate sheath insertable through the vascular system of a subject to bring a distal end of the sheath to a target site in the vascular system; and a filter arrangement configured to extend from the distal end of the sheath, wherein: the filter arrangement comprises a porous membrane switchable between a radially contracted state and a radially expanded state, wherein the radially expanded state is such as to span a cross-section of a blood vessel at the target site to block debris generated upstream from travelling past the porous membrane through the porous membrane while allowing blood to flow past the porous membrane through the porous membrane; and a filter actuation system configured to hydraulically switch the porous membrane between the radially contracted state and the radially expanded state.

Thus, an apparatus is provided that uses a hydraulically switchable porous membrane that can be inserted into a target site in a radially contracted state before being switched into a radially expanded state to provide protection against unwanted migration of debris downstream from a transcatheter operation generating the debris. The hydraulic mechanism provides flexibility of operation, high reliability, and is not excessively complex or expensive to implement.

In an embodiment, the filter actuation system comprises one or more elongate drive members, each drive member being hydraulically actuatable to change shape to drive the switching of the porous membrane. Each drive member may define one or more drive lumens configured such that driving fluid in the one or more drive lumens drives the change in shape of the drive member. This approach has been found to allow the porous membrane to be deformed reliably and with a high degree of control over the shape of the porous membrane.

In an embodiment, the one or more drive lumens is or are offset within a cross-section of the drive member relative to a stiffness centre of the drive member along at least a portion of a longitudinal length of the drive member. Offsetting the drive lumen or drive lumens relative to the stiffness centre allows a hydraulically controllable bending moment to be applied using a simple and/or robust structure.

In an embodiment, each of one or more of the drive members comprises a valve coupled to the one or more drive lumens, the valve being configured to regulate a pressure in the one or more drive lumens and/or to allow injection of liquid to the target site from the one or more drive lumens. The drive members can thus have a dual purpose: namely, contributing to the switching of the porous membrane between the radially contracted and radially expanded states, as well as providing conduits for injecting liquid into the vascular system, such as saline, contrast agent, or drugs. The apparatus thus provides wide ranging functionality in a highly compact and easy to use form.

In an embodiment, the apparatus comprises a plurality of the drive members; and the filter actuation system is configured to control pressures of driving fluid in drive lumens of different drive members independently of each other. This provides flexibility for controlling the shapes of the drive members and the associated switching of the porous membrane. For example, by deliberately driving different drive members to distort differently (e.g., using different respective pressures) it is possible to control the overall shape of the porous membrane in the radially expanded state with a high degree of flexibility. This enhanced control may be used for example to personalize actuation of the porous membrane to the particular vascular morphology of the subject that is to undergo the transcatheter operation.

In an embodiment, a set of drive lumens in multiple different drive members are fluidically connected to each other such that the shapes of the drive members can be controlled in unison by controlling a common pressure in the set of drive lumens. This approach may simplify construction and operation.

In an embodiment, the elongate sheath is switchable between a radially contracted state and a radially expanded state. The sheath in the radially expanded state may define a central lumen for allowing insertion through the central lumen to the target site of a device for performing a transcatheter operation. The elongate sheath can thus adopt a radially contracted state to enable efficient insertion into the subject and then be switched into a radially expanded state to allow devices to be fed through a central lumen of the sheath to perform a transcatheter operation. Multiple functions can thus be performed without requiring multiple corresponding incisions in the subject and associated operational complexity and added trauma.

In an embodiment, the sheath comprises a tubular body defining the internal lumen, the body having a cross-sectional profile in which the stiffness of the body varies as a function of azimuthal angle to promote folding inwards of the body in one or more predetermined ranges of azimuthal angle, the body being configured to be more folded inwards in the one or more ranges when the sheath is in the radially contracted state than when the sheath is in the radially expanded state. This approach provides the required functionality while maintaining reliable operation and a robust structure.

According to an alternative aspect, there is provided a method of providing embolic protection during a transcatheter operation, comprising: inserting an elongate sheath through the vascular system of a subject to bring a distal end of the sheath to a target site in the vascular system, wherein a porous membrane in a radially contracted state extends from the distal end of the sheath during the insertion; and switching the porous membrane into a radially expanded state such that the porous membrane spans a cross-section of a blood vessel at the target site to block debris generated upstream from travelling past the porous membrane through the porous membrane while allowing blood to flow past the porous membrane through the porous membrane, wherein: the switching of the porous membrane from the radially contracted state to the radially expanded state is performed hydraulically.

Embodiments of the disclosure will now be further described, merely by way of example, with reference to the accompanying drawings.

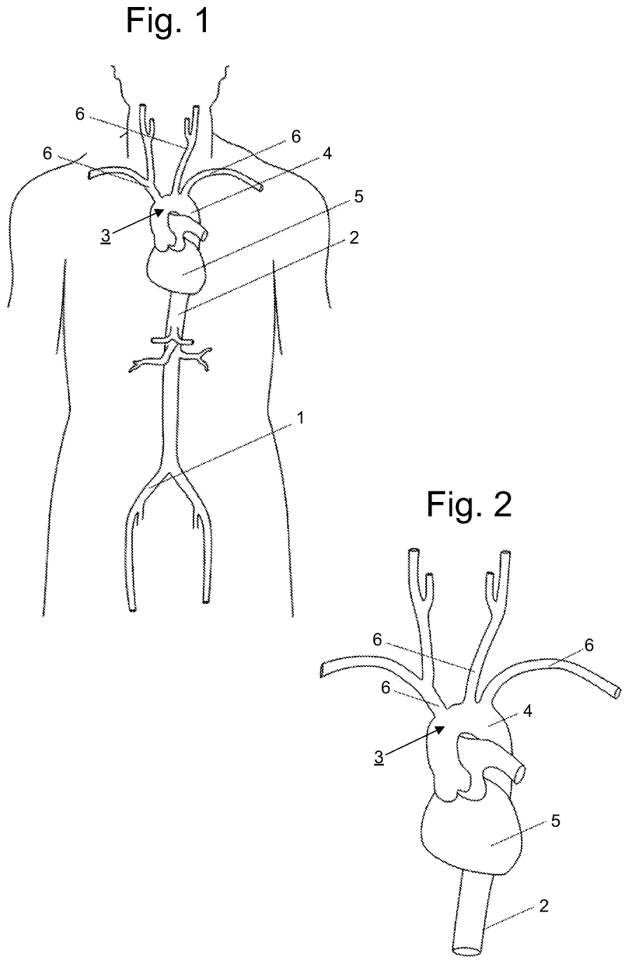

FIGS. 1 and 2 schematically depict features of the human anatomy relevant to apparatus and methods of the present disclosure.

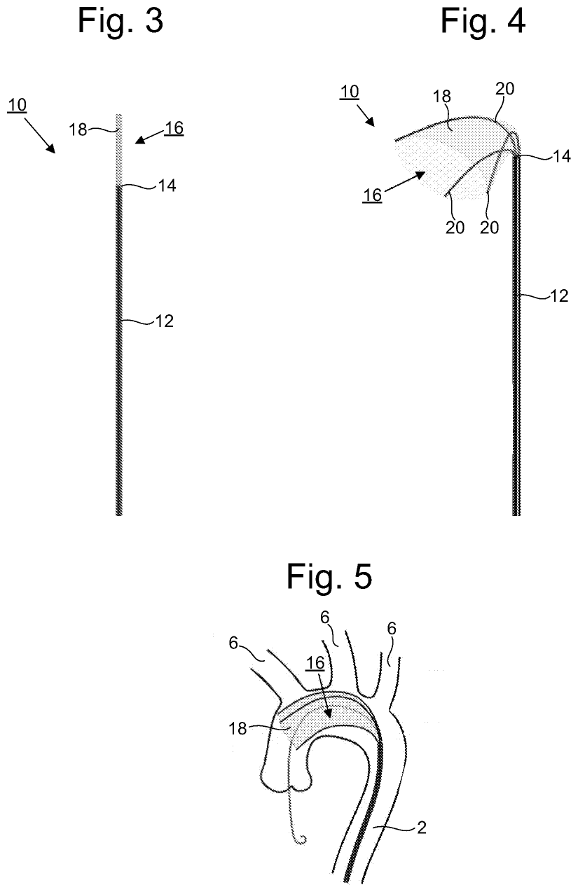

FIG. 3 depicts an apparatus for providing embolic protection having a sheath and a porous membrane in a radially contracted state.

FIG. 4 depicts the apparatus of FIG. 3 with the porous membrane in a radially expanded state.

FIG. 5 depicts deployment of the apparatus of FIGS. 3 and 4 at a target site in the aortic arch.

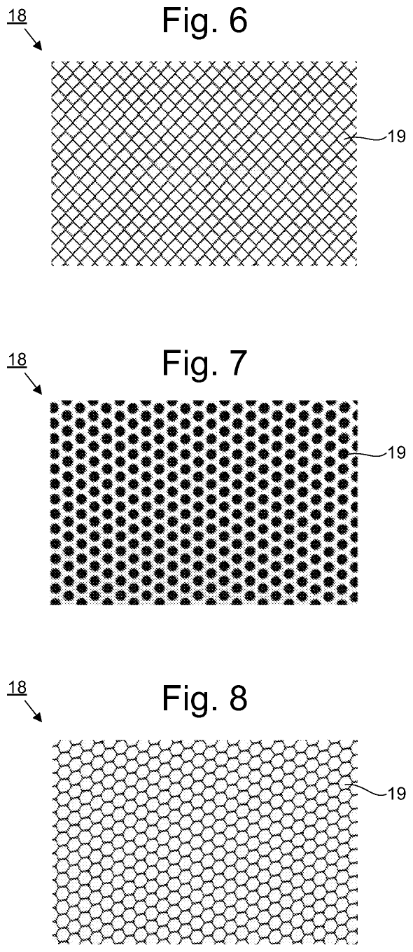

FIGS. 6 to 8 depict example pore structures of the porous membrane 18.

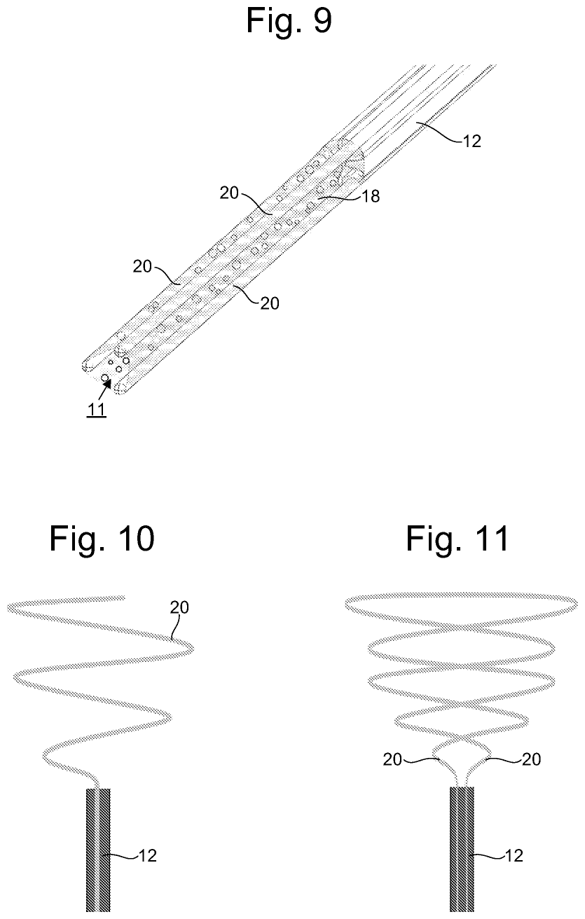

FIG. 9 is a perspective view of a distal end of an apparatus for providing embolic protection showing a porous membrane and associated drive members in a radially contracted state.

FIG. 10 depicts example actuation of a drive member into a spiral.

FIG. 11 depicts example actuation of two drive members into spirals.

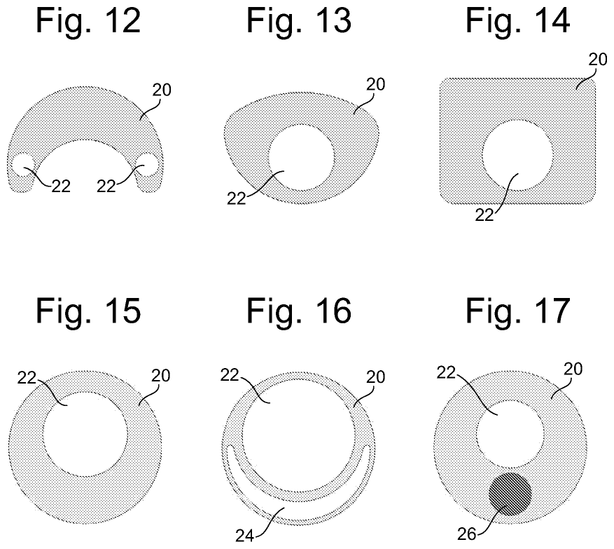

FIGS. 12-17 depict example cross-sections for drive members.

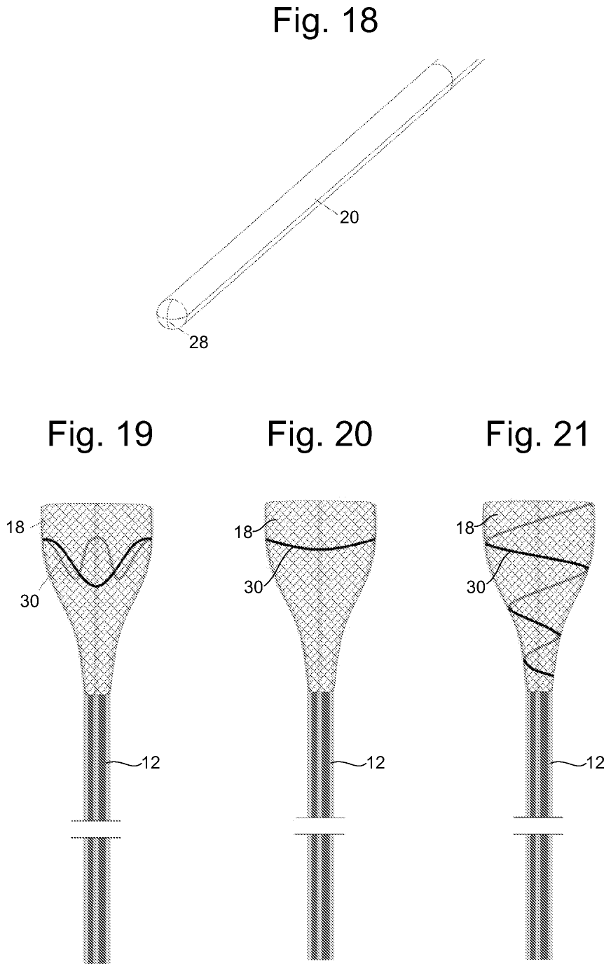

FIG. 18 is a perspective view of a distal end of a drive member.

FIGS. 19 to 21 depict example configurations for circumferentially extending support wires for mechanically supporting the porous membrane.

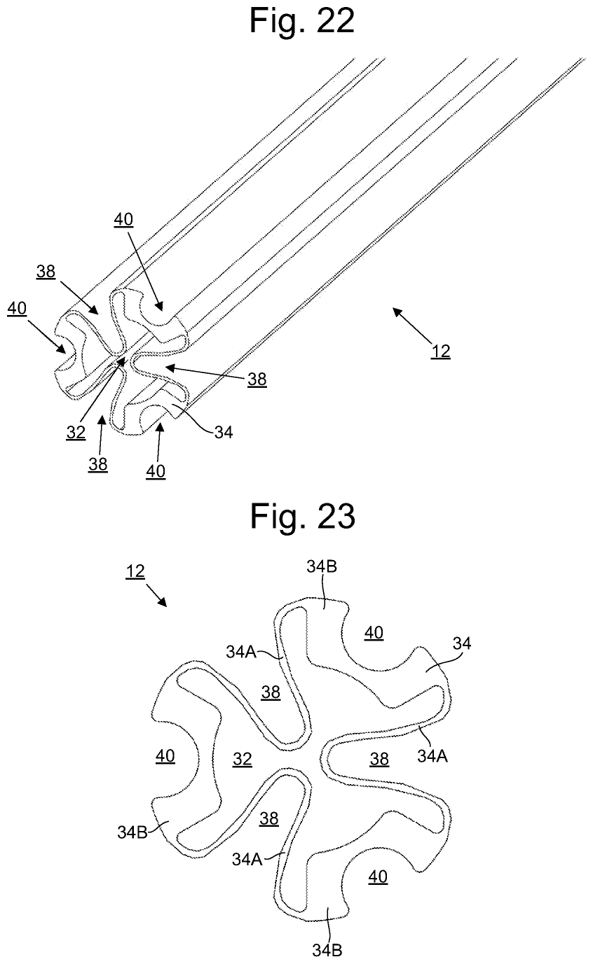

FIG. 22 is a perspective view of a distal end of a sheath in a radially contracted state.

FIG. 23 is a cross-sectional end view of the sheath of FIG. 22.

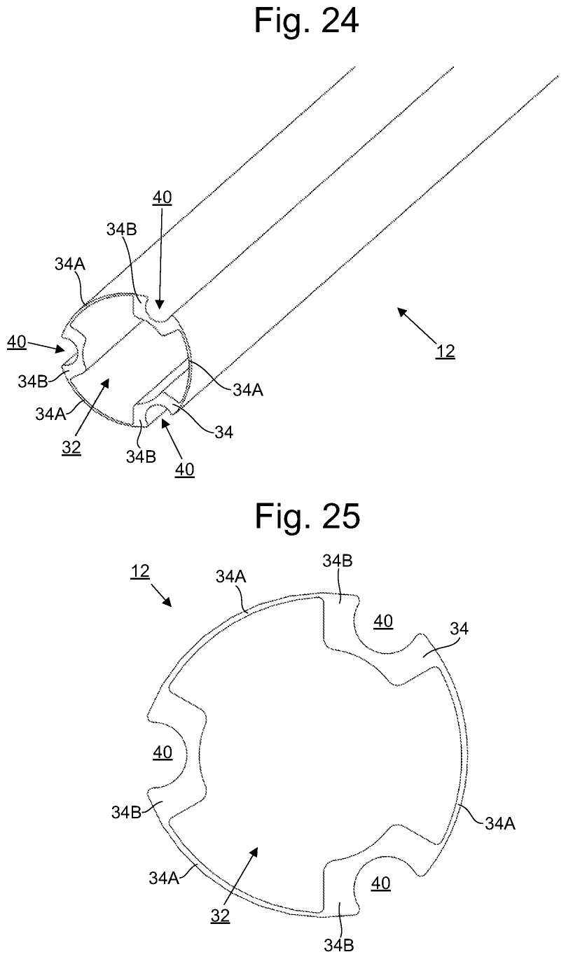

FIG. 24 is a perspective view of a distal end of a sheath in a radially expanded state.

FIG. 25 is a cross-sectional end view of the sheath of FIG. 24.

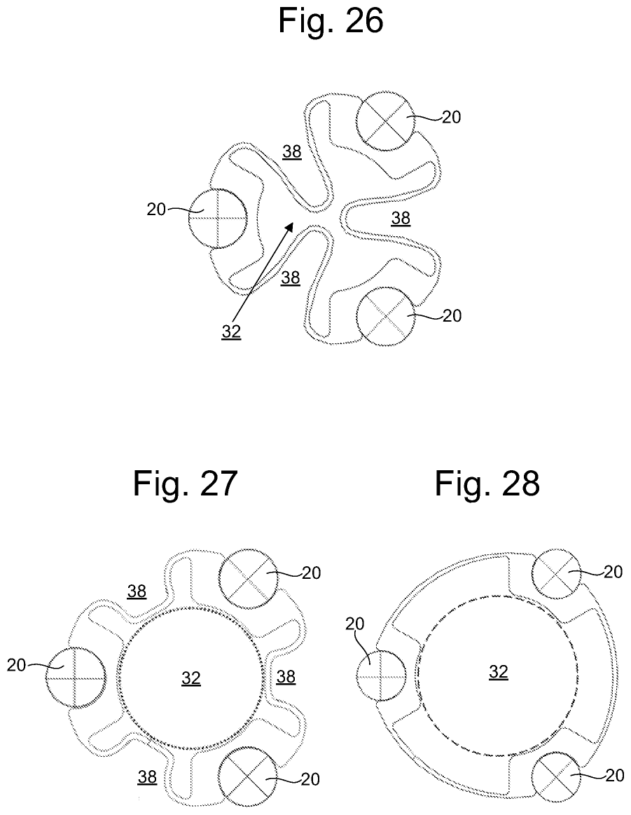

FIG. 26 is cross-sectional end view of the sheath in the radially contracted state with drive members received in drive member grooves.

FIG. 27 is cross-sectional end view of the sheath of FIG. 26 in a first radially expanded state.

FIG. 28 is cross-sectional end view of the sheath of FIG. 26 in a second radially expanded state.

FIGS. 29 to 40 depict example method steps in the context of a surgical operation comprising introduction of a prosthetic valve into a subject's native aortic valve.

The present disclosure relates to methods and apparatus 10 for providing embolic protection during a transcatheter operation. The transcatheter operation may comprise any cardiac operation that may generate potentially problematic (e.g., embolism causing) debris, including for example deployment of a prosthesis such as an aortic valve prosthesis or aortic valve valvuloplasty.

FIGS. 1 and 2 depict relevant features of the human anatomy. FIGS. 3 and 4 depict an example apparatus 10. The apparatus 10 comprises an elongate sheath 12. The sheath 12 is insertable through the vascular system of a subject to bring a distal end 14 of the sheath 12 to a target site 3 in the vascular system. The target site 3 may be in the aorta. Referring to FIG. 1, the sheath 12 may, for example, be inserted via transfemoral access 1 through the descending aorta 2 to a target site 3 in, or in the vicinity of, the aortic arch 4 above the heart 5. The target site 3 may be adjacent to arteries 6, including the brachiocephalic artery, left common carotid artery and left subclavian artery. The sheath 12 may thus be sufficiently long to extend for example from the upstream iliac bifurcation of the abdominal aorta to the aortic arch.

The apparatus 10 comprises a filter arrangement 16. The filter arrangement 16 is configured to extend from the distal end 14 of the sheath 12. In some arrangements, the filter arrangement 16 is attached, for example rigidly attached, to the distal end 14 of the sheath 12.

The filter arrangement 16 comprises a porous membrane 18. The porous member 18 is switchable between a radially contracted state and a radially expanded state. The filter arrangement 16 is thus configured to allow switching of the porous membrane 18 between a radially contracted state and a radially expanded state.

FIG. 3 depicts the porous membrane 18 in the radially contracted state. In an arrangement, the radially contracted state is such as to allow the sheath 12 and the filter arrangement 16 to be inserted through the vascular system (e.g., from the transfemoral access 1 to the target site 3 in the aortic arch 4) while the porous membrane 18 is in the radially contracted state.

FIG. 4 depicts the porous membrane 18 in the radially expanded state. The radially expanded state may be such as to span a cross-section of a blood vessel at the target site 3 to block debris generated upstream, for example by a transcatheter operation, from travelling past the porous membrane 18 through the porous membrane 18 (i.e., through pores of the porous membrane 18) while allowing blood to flow past the porous membrane 18 through the porous membrane 18 (i.e., through pores of the porous membrane 18).

FIG. 5 schematically depicts a distal portion of the apparatus 10 deployed in the aortic arch. In this example, debris generated below the distal end of the porous membrane 18 in the left part of the figure will be prevented by the porous membrane 18 from being carried past the porous membrane 18 into any of the arteries 6 or down the descending aorta 3. Small gaps depicted between the porous membrane 18 and adjacent walls of the vascular system in FIG. 5 may not exist in practice or may be small enough that debris of concern will not be able to pass through the gaps. The porous membrane 18 may, for example, be in contact with the adjacent walls, optionally continuously along a closed loop path, and/or press radially outwardly against the walls to minimise or remove any path past the porous membrane 18 that does not involve passing through the porous membrane 18 (e.g., through pores in the porous membrane 18). The porous membrane 18 in the radially expanded state may have a frusto-conical form or similar, with an opening at the distal end that is larger than an opening at a proximal end.

The porous membrane 18 may take various forms. The porous membrane 18 may for example comprise a porous mesh material such as a fabric of knitted, braided, woven, or nonwoven fibres, filaments, or wires. The porous membrane 18 may comprise pores 19. Sizes of the pores 19 may be chosen to prevent emboli over a predetermined size from passing therethrough. In some arrangements, a pore size of at least a subset of the pores 19 is in the range of about 50-250 μm when the porous membrane 18 is in the radially expanded state. The pores 19 may be sized to allow other catheters, such as a contrast catheter, to pass through the pores 19, for example from the brachiocephalic artery, left common carotid artery, or left subclavian artery. Example pore structures for the porous membrane 18 are depicted in FIGS. 6-8.

In some arrangements, the porous membrane 18 is configured to be resilient. The porous membrane 18 may comprise a fabric made of a resilient metal, a polymer material, a malleable material, a plastically deformable material, a shape-memory material, or combinations thereof. In some arrangements, the porous membrane 18 comprises an anti-thrombogenic coating.

Referring to FIG. 9, when it is time to withdraw the apparatus 10, the porous membrane 18 can be switched back into the radially contracted state. Any debris 11 collected in the membrane 18 is retained safely in the porous membrane 18 and can be removed from the body along with the porous membrane 18.

The apparatus 10 comprises a filter actuation system configured to hydraulically switch the porous membrane 18 between the radially contracted state and the radially expanded state. In some arrangements, the filter actuation system comprises one or more elongate drive members 20. Each drive member 20 is hydraulically actuatable to change shape to drive the switching of the porous membrane 18. Each of one or more of the drive members 20 may, for example, be provided radially inwardly of the porous membrane 18 such that radial expansion of the drive member 20 (e.g., splaying outwards) pushes the porous membrane 18 outwards from the inside (e.g., into a cone-like shape). Alternatively, each of one or more of the drive members 20 may be provided radially outside of the porous membrane 18 but mechanically connected thereto, such that radial expansion of the drive member 20 (e.g., splaying outwards) pulls the porous membrane 18 outwards from the outside (e.g., into a cone-like shape). The porous membrane 18 may be resiliently biased in a radially inwards direction to hold the drive members 20 in a radially contracted resting (unactuated) state. When no hydraulic driving force is applied to the drive members 20 the porous membrane 18 may hold them in the radially contracted state (e.g., positioned close to each other over their whole longitudinal lengths).

Each drive member 20 may extend along the whole or a majority of the length of the sheath 12. Each drive member 20 may for example be configured to allow liquid to be driven into the drive member 20 from outside of the subject to hydraulically actuate the drive member 20 while the distal end 14 of the sheath is at the target site 3. Each drive member 20 may for example extend from a haemostatic valve allowing injection of liquid into the driving member 20 through to the target site 3. The haemostatic valve may be located for example near the upstream iliac bifurcation of the abdominal aorta. Each drive member 20 may be further configured to extend beyond the distal end 14 of the sheath 12. A portion (which may be referred to as an “extending portion”) of each drive member 20 that extends beyond the distal end 14 may have a length in the range of about 50 mm to about 150 mm. The extending portion of the drive member 20 may be configured to extend for example from roughly a level of transition between the descending aorta and the aortic arch into the ascending aorta. The change in shape of the drive member 20 caused by the hydraulic actuation may comprise or consist of a change in the locus of a longitudinal axis of the extending portion of the drive member 20. In some arrangements, the longitudinal axis of the extending portion of the drive member 20 is substantially linear when the porous membrane 18 is in the radially contracted state (as exemplified in FIG. 9) and is hydraulically actuatable to be non-linear when the porous membrane 18 is in the radially expanded state. The non-linear form is exemplified in FIG. 4 where the drive members 20 are driven to splay radially outwards from the distal end 14 of the sheath 12. Other non-linear forms are possible, such as depicted in FIGS. 10 and 11. For example, an extending portion of a drive member 20 may be configured to deform from a linear state into a spiral. This may allow a single drive member 20, or a relatively small number of drive members 20 such as two drive members 20, to contribute effectively to expanding the porous membrane 18 into the radially expanded state and/or mechanically supporting the porous membrane 18 in the radially expanded state through all azimuthal angles. The porous membrane 18 is omitted from FIGS. 10 and 11 for clarity but would be understood to be present in practice. FIG. 10 depicts an example using a single drive member 20 that deforms into a spiral. FIG. 11 depicts an example using two drive members 20 that deform into spirals having opposite spiral orientations (i.e., spirals that spiral in opposite senses when viewed along a common axis of the spirals).

In some arrangements, as exemplified in FIGS. 4 and 9, the apparatus 10 comprises a plurality of the drive members 20. In such a case, the drive members 20 may be configured such that the extending portions of the drive members 20 are substantially aligned with each other (e.g., in linear form as exemplified in FIG. 9) parallel to a longitudinal axis of the sheath 12 at the distal end 14 when the porous membrane 18 is in the radially contracted state, and radially diverge from each other in the distal direction (i.e., getting further apart as a function of increasing distance away from an insertion point of the apparatus 10 into the subject) when the porous membrane 18 is in the radially expanded state. The plurality of drive members 20 may, for example, be driven to splay outwards as exemplified in FIG. 4.

In some arrangements each drive member 20 is resilient such that when the drive member 20 is unactuated the drive member 20 remains in, or returns to, a state corresponding to the porous membrane 18 being in the radially contracted state (e.g., into a state in which the longitudinal axis of the extending portion of the drive member 20 is substantially linear).

Each drive member 20 defines one or more drive lumens 22 configured such that driving fluid in the one or more drive lumens (e.g., driving fluid such as a liquid into the one or more drive lumens and/or changing a pressure of fluid such as a liquid in the one or more drive lumens) drives the change in shape of the drive member 20. In each drive lumen 22, hydraulic pressure will generate an axial force inside the drive lumen 22. By suitably configuring the shape and/or composition of the cross-section of the drive member 20, including the shapes, sizes and/or positions of the one or more drive lumens 22, it is possible to determine in advance how the shape of the drive member 20 will respond to the hydraulic pressure. For example, it is possible to ensure that the drive member 20 will deform in a way that promotes a desired switching of the porous membrane 18 between the radially contracted state and the radially expanded state. For example, the one or more drive lumens 22 may be offset within a cross-section of the drive member 20 relative to a stiffness centre of the drive member 20 along at least a portion of a longitudinal length of the drive member 20 (e.g., including at least the extending portion of the drive member 20 that extends beyond the sheath 12). The cross-section of the drive member 20 may thereby be designed to have a stiffness centre that is eccentric relative to a force centre defined by the drive lumen 22 or drive lumens 22 defined in the drive member 20. This eccentricity causes the drive member 20 to deform into a designed deployment shape (e.g., corresponding to the porous membrane 18 being in the radially expanded state) from a compact form (e.g., axially aligned and/or corresponding to the porous membrane 18 being in the radially contracted state). The eccentricity between the force centre and stiffness centre induces a moment that deforms the drive member 20. The deformation induced in each drive member 20 can be configured to cause the porous membrane 18 to adopt a wide range of shapes, including a cone or cone-like shape (e.g., a frustocone), a tube or tube-like shape, a spiral, or any other form that creates space to allow one or more other working catheters to pass through a proximal opening in the porous membrane 18 while splaying out in a manner suitable to capture all or a large proportion of debris generated by a surgical operation upstream of the porous membrane 18.

FIGS. 12-17 depict example cross-sections for the drive member 20. The drive member can be made of single or multiple materials such as thermoplastic polyurethane, polyethylene terephthalate, Pebax, or other biocompatible polymers or elastomers. For the scenarios in which the drive member 20 is made of a single material, the cross-section may typically be designed to be asymmetric (e.g., not cylindrically symmetric). This may be achieved by providing a non-cylindrically symmetric outer shape, as exemplified in FIGS. 12-14, and/or by providing cylindrically asymmetric structure or cavities inside the drive member 20, as exemplified in FIG. 16. Alternatively or additionally, where the drive member 20 is formed from multiple materials, the drive member 20 could be symmetrically arranged relative to the drive lumen 22 or drive lumens 22 (e.g., with the drive member 20 and a drive lumen 22 being axially aligned with each other), with the eccentricity being provided instead by an asymmetrical distribution of the different materials in the cross-section. An example configuration using multiple materials is shown in FIG. 17. Here, the portion of the cross-section labelled 20 consists of a first material and the portion of the cross-section labelled 26 consists of a second material, embedded in the first material and having different stiffness properties to the first material. Stiffness properties and cross-sectional shapes of the first and second materials are designed to ensure that the force centre of the drive lumen 22 and the stiffness centre of the drive member 20 do not overlap.

As exemplified in FIG. 18, in some arrangements each of one or more of the drive members 20 comprises a valve 28 (e.g., a one-way valve) coupled to the one or more drive lumens 22. The valve 28 may be configured to regulate a pressure in the one or more drive lumens 22 and/or to allow injection of liquid to the target site 3 from the one or more drive lumens 22, such as injection of saline, a contrast agent, or a drug agent. The valve 28 may be configured to open when the pressure in a drive lumen 22 exceeds a predetermined threshold pressure for example.

In some arrangements, the filter actuation system is configured to control pressures of driving fluid in drive lumens 22 of different drive members 20 independently of each other. This may provide more flexibility for controlling the shapes of the drive members 20 and the associated switching of the porous membrane 18. For example, by deliberately driving different drive member 20 to distort differently (e.g., using different respective pressures) it is possible to control the overall shape of the porous membrane 18 in the radially expanded state with a high degree of flexibility. This enhanced control may be used for example to personalize actuation of the porous membrane 18 to the particular vascular morphology of the subject that is to undergo the transcatheter operation. Alternatively or additionally, a set of drive lumens 22 in multiple different drive members 20 may be fluidically connected to each other such that the shapes of the drive members 20 can be controlled in unison by controlling a common pressure in the set of drive lumens 22. This approach may simplify construction and operation.

In some arrangements, each of one or more of the drive members 20 further defines a supplementary lumen 24. The supplementary lumen 24 is fluidically isolated from a drive lumen 22 in the drive member 20. The supplementary lumen 24 is configured to allow injection of liquid to the target site 3 through the supplementary lumen 24, such as injection of saline, a contrast agent, or a drug agent. An example of such a supplementary lumen 24 is shown in FIG. 16. One of the two drive lumens 22 shown in FIG. 12 could also be configured to operate and/or be used as a supplementary lumen. A valve (e.g., a one-way valve) may or may not be provided at a distal end of the supplementary lumen 24.

In some arrangements, the filter arrangement comprises one or more resilient support wires 30 mechanically connected to the porous membrane 18. Each support wire 30 may promote mechanical stability and/or strength of the porous membrane 18 in the radially expanded state. The support wire 30 may for example comprise or consist of stainless steel or nitinol. The support wire 30 can be embedded into the drive member 20. The region 26 of second material shown in FIG. 17 could be a support wire for example. The support wire 30 can be configured to tune the flexibility or control the motion of the drive member 20 used to actuate the porous membrane 18. Alternatively or additionally, the support wire 30 may advantageously provide mechanical support to the sheath 12, which may assist in providing the sheath 12 with a suitable rigidity for navigating through the vasculature system from an access point (e.g., transfemoral access 1) to the target site 3. Alternatively or additionally, the support wire 30 can be connected to the porous membrane 18 separately from the drive members, such as circumferentially (e.g., to form a closed loop or spiral around an axis of the porous membrane 18). The support wire 30 in such arrangements could comprise polymer or metallic wires or webbing. The support wire may enhance the strength and/or radial resilience of the porous membrane 18 in the radially expanded state, thereby improving the stability of deployment at the target site 3 and/or reliable protection against debris bypassing the porous membrane 18 through unwanted gaps between the porous membrane and walls of the local vasculature. Non-limiting example configurations are depicted in FIGS. 19 to 21.

In some arrangements, the sheath 12 is switchable between a radially contracted state and a radially expanded state. An example sheath 12 in the radially contracted state is depicted in FIGS. 22 and 23. An example sheath 12 in the radially expanded state is depicted in FIGS. 24 and 25. The sheath 12 in the radially contracted state may define a central lumen 32 that is relatively small in cross-section. This allows an overall outer dimension of the sheath 12 to be relatively small, thus facilitating insertion and advancement of the sheath 12 into a subject and through the vascular system to a target site 3. The central lumen 32 may, however, be large enough to accommodate a guide wire 44 even when the sheath 12 is in the radially contracted state.

The sheath 12 in the radially expanded state may define a central lumen 32 that is significantly larger. The larger central lumen 32 may be suitable, for example, to allow insertion of a device through the central lumen 32 to the target site 3 for performing a transcatheter operation. For example, the sheath 12 may be configured to have an outer diameter in the radially contracted state that is smaller than an inner diameter of the central lumen 32 in the radially expanded state.

The porous membrane 18 in the radially expanded state may comprise a proximal opening leading to the central lumen 32 and a distal opening that is larger than the proximal opening 32. The central lumen 32 thus opens out distally into a region within the porous membrane 18 (e.g., within the cone-like shape defined by the porous membrane 18).

The sheath 12 may be configured to self-expand from the radially contracted state to the radially expanded state. Alternatively or additionally, the sheath 12 may be configured to be expanded by longitudinal movement through the sheath 12 of the device for performing the transcatheter operation. As depicted in FIGS. 22-25, the sheath 12 may comprise a tubular body 34 defining the internal lumen 32. The body 34 may have a cross-sectional profile in which a stiffness of the body 34 varies as a function of azimuthal angle to promote folding inwards of the body 34 in one or more predetermined ranges of azimuthal angle. The body 34 may be configured to be more folded inwards in the one or more ranges when the sheath 12 is in the radially contracted state than when the sheath 12 is in the radially expanded state. The variation of the stiffness of the body 34 with azimuthal angle may at least partially be defined by a corresponding variation in radial thickness of the body 34 with azimuthal angle.

In the example shown in FIGS. 22 and 23, the body 34 is configured to fold inwards in three azimuthal regions to form three respective folds 38 when the sheath 12 is in the radially contracted state. The radial thickness of the body 34 of the sheath 12 can be seen to be smaller in each of the three azimuthal regions corresponding to the folds 38 than elsewhere around the circumference of the sheath 12. For example, the radial thickness of the wall portions labelled 34A corresponding to the folds 38 can be seen to be smaller than the radial thickness of the walls portions labelled 34B that do not fold inwards.

In some arrangements, the sheath 12 defines one or more drive member grooves 40 in an outer surface of the sheath 12. Each drive member groove 40 is configured to receive a respective one of the drive members 20. Each drive member groove 40 may be formed in a range of azimuthal angles (e.g., corresponding to thicker wall portions 34B) in which the body 34 of the sheath 12 has a stiffness that is higher than in the ranges of azimuthal angle (e.g., corresponding to thinner wall portions 34A) configured to promote the folding inwards of the body 34.

FIGS. 26 to 28 depict example arrangements of the sheath 12 with three drive members 20 positioned in respective grooves 40 that are separated from each other by 120 degrees. FIG. 26 shows an arrangement with the sheath 12 in a radially contracted state. FIG. 27 shows an arrangement with the sheath 12 in a first radially expanded state, which is suitable for accommodating a first device, such as an introducer (represented schematically by the dotted line circle) in the central lumen 32. FIG. 28 shows an arrangement with the sheath 12 in a second radially expanded state, radially larger than the first radially expanded state, which is suitable for accommodating a second device, such as a prosthetic valve (represented schematically by the dashed line circle) in the central lumen 32. The second device (e.g., prosthetic valve) may be radially larger than the first device (e.g., introducer). The sheath 12 is thus capable of radially expanding to different extents to accommodate a range of differently sized devices in the central lumen 32.

Any of the apparatus arrangements disclosed herein may be used in a method of providing embolic protection during a surgical operation. The method may comprise inserting an elongate sheath 12 through the vascular system of a subject to bring a distal end 14 of the sheath 12 to a target site 3 in the vascular system. A porous membrane 18 in a radially contracted state may extend from the distal end 14 of the sheath 12 during the insertion. The method comprises switching the porous membrane 18 into a radially expanded state such that the porous membrane 18 spans a cross-section of a blood vessel at the target site 3 to block debris generated upstream from travelling past the porous membrane 18 through the porous membrane 18 while allowing blood to flow past the porous membrane 18 through the porous membrane 18. The switching of the porous membrane 18 from the radially contracted state to the radially expanded state is performed hydraulically, for example using any of the apparatus elements and techniques discussed above. The method may further comprise introducing a device for performing the transcatheter operation through a central lumen 32 of the sheath 12. The method may further comprise performing the transcatheter operation using the device while the porous membrane 18 is in the radially expanded state. The method may further comprise trapping debris generated by the transcatheter operation in the porous membrane 18 by switching the porous membrane 18 from the radially expanded state to the radially contracted state after the performing of the transcatheter operation. The sheath 12 and porous membrane 18 may subsequently be removed from the subject.

Example method steps are depicted in FIGS. 29-40 in the context of a surgical operation comprising introduction of a prosthetic valve 42 into a subject's native aortic valve.

In a first step, as depicted in FIG. 29, a guide wire 44 is introduced into the subject's vascular system and fed to and through the aortic arch 4 to a region adjacent to a surgical site of interest, in this case adjacent to the aortic valve.

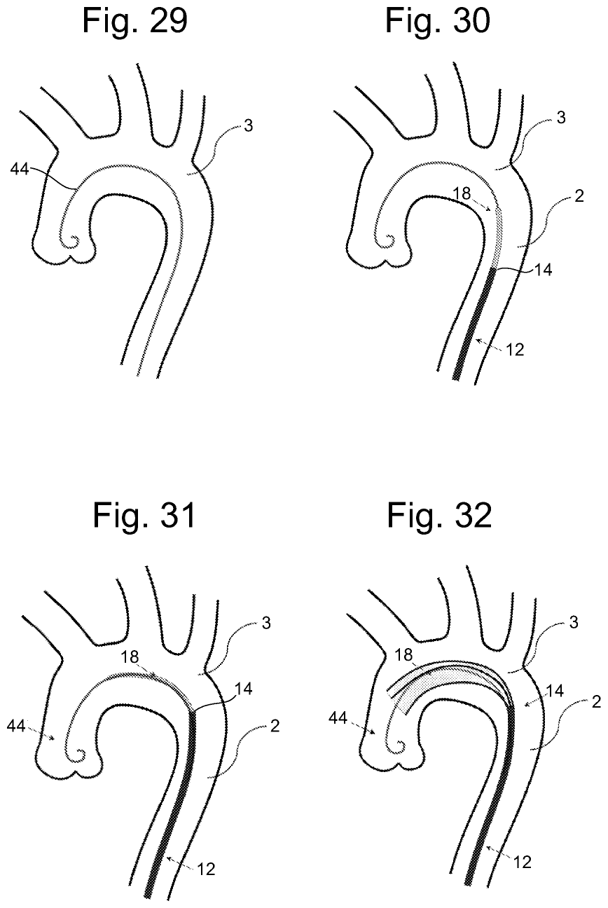

Subsequently, as depicted in FIGS. 30 and 31, the sheath 12 is advanced along the guide wire 44 from the upstream iliac bifurcation of the abdominal aorta through the descending aorta 2 to the target site 3. The porous membrane 18 extends from the distal end 14 of the sheath 12 during the advancement. The porous membrane 18 is in the radially contracted state during this phase.

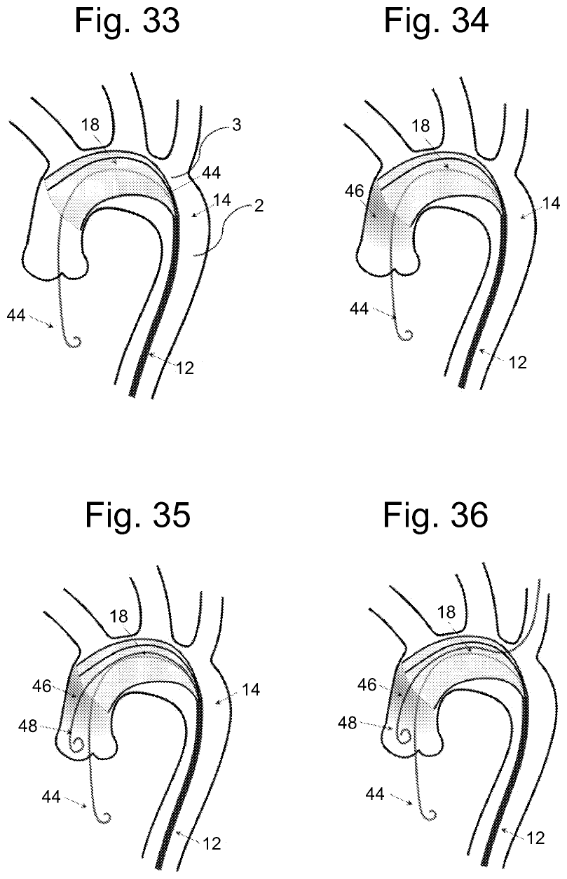

Subsequently, as depicted in FIGS. 32 and 33, the porous membrane 18 is switched from the radially contracted state to the radially expanded state. When fully radially expanded (as shown in FIG. 33), the porous membrane 18 spans a cross-section of a blood vessel at the target site 3, forming a fit against the inner wall of the blood vessel. Subsequently, contrast agent 46 may be injected into the target site 3, for example via one or more of the drive members 20 (as depicted in FIG. 34) or via a separate catheter 48, which may be inserted for example via the transfemoral artery and follow the length of the sheath 12 (as depicted in FIG. 35) or via the left subclavian artery (as depicted in FIG. 36). Access via the transfemoral artery may be implemented for example by providing a self-sealing inlet section in the sheath 12 to allow the separate catheter 48 to access the sheath from the transfemoral artery. Access via the left subclavian artery may be implemented by pushing the catheter 48 through suitably sized micropores in the porous membrane 18.

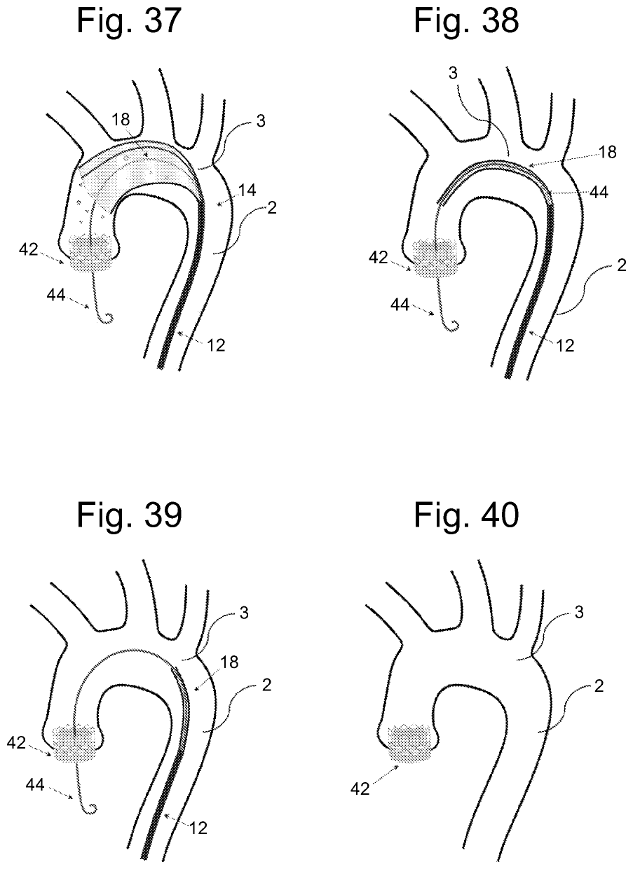

Subsequently, the prosthetic valve 42 is deployed after the contrast agent has made the aortic valve visible, as depicted in FIG. 37. Debris start to release and travel along the blood flow into the porous membrane 18, which is in the radially expanded state.

Once all the debris particles are collected inside the porous membrane 18, the porous membrane 18 is switched from the radially expanded state back into the radially contracted state, as depicted in FIG. 38. The radial contraction of the porous membrane 18 traps the debris in the porous membrane 18. The sheath 12 and porous membrane 18 can then be withdrawn from the subject as depicted in FIG. 39, safely removing the debris. Finally, the guide wire 44 is removed as depicted in FIG. 40.

1. An apparatus for providing embolic protection during a transcatheter operation, the apparatus comprising:
an elongate sheath insertable through the vascular system of a subject to bring a distal end of the sheath to a target site in the vascular system; and
a filter arrangement configured to extend from the distal end of the sheath, wherein:
the filter arrangement comprises a porous membrane switchable between a radially contracted state and a radially expanded state, wherein the radially expanded state is such as to span a cross-section of a blood vessel at the target site to block debris generated upstream from travelling past the porous membrane through the porous membrane while allowing blood to flow past the porous membrane through the porous membrane; and
a filter actuation system configured to hydraulically switch the porous membrane between the radially contracted state and the radially expanded state.

2. The apparatus of claim 1, wherein the filter actuation system comprises one or more elongate drive members, each drive member being hydraulically actuatable to change shape to drive the switching of the porous membrane.

3. The apparatus of claim 2, wherein a longitudinal axis of a portion of each drive member extending beyond the distal end of the sheath is substantially linear when the porous membrane is in the radially contracted state and is hydraulically actuatable to be non-linear when the porous membrane is in the radially expanded state.

4. The apparatus of claim 2, wherein:
the apparatus comprises a plurality of the drive members; and
the drive members are configured such that portions of the drive members extending beyond the distal end of the sheath are substantially aligned with each other parallel to a longitudinal axis of the sheath at the distal end when the porous membrane is in the radially contracted state, and radially diverge from each other in the distal direction when the porous membrane is in the radially expanded state.

5. The apparatus of claim 2, wherein each drive member defines one or more drive lumens configured such that driving fluid in the one or more drive lumens drives the change in shape of the drive member.

6. The apparatus of claim 5, wherein the one or more drive lumens is or are offset within a cross-section of the drive member relative to a stiffness centre of the drive member along at least a portion of a longitudinal length of the drive member.

7. The apparatus of claim 5, wherein each of one or more of the drive members comprises a valve coupled to the one or more drive lumens, the valve being configured to regulate a pressure in the one or more drive lumens and/or to allow injection of liquid to the target site from the one or more drive lumens.

8. The apparatus of claim 5, wherein:
the apparatus comprises a plurality of the drive members; and
the filter actuation system is configured to control pressures of driving fluid in drive lumens of different drive members independently of each other.

9. The apparatus of claim 5, wherein:
the apparatus comprises a plurality of the drive members; and
a set of drive lumens in multiple different drive members are fluidically connected to each other such that the shapes of the drive members can be controlled in unison by controlling a common pressure in the set of drive lumens.

10. The apparatus of claim 5, wherein each of one or more of the drive members further defines a supplementary lumen, the supplementary lumen being fluidically isolated from a drive lumen in the drive member and being configured to allow injection of liquid to the target site from the supplementary lumen.

11. The apparatus of claim 1, wherein the porous membrane is resilient and, optionally, biased in a radially inwards direction.

12. The apparatus of claim 1, wherein the filter arrangement comprises one or more resilient support wires mechanically connected to the porous membrane.

13. The apparatus of claim 1, wherein the elongate sheath is switchable between a radially contracted state and a radially expanded state.

14. The apparatus of claim 13, wherein the sheath in the radially expanded state defines a central lumen for allowing insertion through the central lumen to the target site of a device for performing a transcatheter operation.

15. The apparatus of claim 14, wherein the porous membrane in the radially expanded state comprises a proximal opening leading to the central lumen and a distal opening that is larger than the proximal opening.

16. The apparatus of claim 14, wherein the sheath is configured to self-expand and/or is configured to be expanded by longitudinal movement through the sheath of the device for performing the transcatheter operation.

17. The apparatus of claim 13, wherein the sheath comprises a tubular body defining the internal lumen, the body having a cross-sectional profile in which a stiffness of the body varies as a function of azimuthal angle to promote folding inwards of the body in one or more predetermined ranges of azimuthal angle, the body being configured to be more folded inwards in the one or more ranges when the sheath is in the radially contracted state than when the sheath is in the radially expanded state.

18. The apparatus of claim 17, wherein the variation of the stiffness of the body with azimuthal angle is at least partially defined by a corresponding variation in radial thickness of the body with azimuthal angle.

19. The apparatus of claim 17, wherein the sheath defines one or more drive member grooves in an outer surface of the sheath, each drive member groove being configured to receive a respective one of the drive members.

20. The apparatus of claim 19, wherein each drive member groove is formed in a range of azimuthal angles in which the body of the sheath has a stiffness that is higher than in the ranges of azimuthal angles configured to promote the folding inwards of the body.

21. The apparatus of claim 13, wherein an outer diameter of the sheath in the radially contracted state is less than an inner diameter of the central lumen in the radially expanded state.

22. The apparatus of claim 1, wherein the target site is in within the aorta of the subject.

23. The apparatus of claim 1, wherein the transcatheter operation comprises a cardiac operation associated with generation of debris, optionally deployment of a prosthesis such as an aortic valve prosthesis or aortic valve valvuloplasty.

24. A method of providing embolic protection during a transcatheter operation, comprising:
inserting an elongate sheath through the vascular system of a subject to bring a distal end of the sheath to a target site in the vascular system, wherein a porous membrane in a radially contracted state extends from the distal end of the sheath during the insertion; and
switching the porous membrane into a radially expanded state such that the porous membrane spans a cross-section of a blood vessel at the target site to block debris generated upstream from travelling past the porous membrane through the porous membrane while allowing blood to flow past the porous membrane through the porous membrane, wherein:
the switching of the porous membrane from the radially contracted state to the radially expanded state is performed hydraulically.

25. The method of claim 24, further comprising:
introducing a device for performing the transcatheter operation through a central lumen of the sheath; and
performing the transcatheter operation using the device while the porous membrane is in the radially expanded state.

26. The method of claim 25, further comprising:
trapping debris generated by the transcatheter operation in the porous membrane by switching the porous membrane from the radially expanded state to the radially contracted state after the performing of the transcatheter operation; and
withdrawing the sheath and porous membrane from the subject.
